(19) United States
(12) Patent Application Publication
(10) Pub. No.: US 2019/0358088 A1
(43) Pub. Date: Nov. 28, 2019

(54) ACTIVATED CARBON COMPOSITE WOUND DRESSING

(71) Applicant: CALGON CARBON CORPORATION, Moon Township, PA (US)

(72) Inventors: Wayne LAVOCAH, South Shields, Durham (GB); Paul CURTIS, Murton, Durham (GB); Robert BROWN, Hesketh Bank, Preston, Lancashire (GB); Dean SLEIGH, Aberford, Leeds, Yorkshire (GB)

(73) Assignee: CALGON CARBON CORPORATION, Moon Township, PA (US)

(21) Appl. No.: 16/472,637

(22) PCT Filed: Dec. 22, 2017
(86) PCT No.: PCT/US2017/068193

§ 371 Date: Jun. 21, 2019

Related U.S. Application Data
(60) Provisional application No. 62/438,651, filed on Dec. 23, 2016.

(51) Int. Cl. A61F 13/00; C09J 11/04; C09J 139/06; C08K 3/04; A61F 13/02; A61L 15/18

(52) U.S. Cl. CPC A61F 13/00008; C09J 11/04; C09J 139/06; A61L 15/18; A61F 13/00029; A61F 13/0206; C08K 3/04

(57) ABSTRACT

Composite wound dressings including an activated carbon material, a barrier material layer, a liquid-absorbing material layer and an outer liquid-impermeable layer are disclosed herein. The composite wound dressings include a bond substantially bonding a portion of the liquid-impermeable outer layer and the barrier material layer to the activated carbon containing layer, the bond forming a sealed pouch of un-bonded layer of the composite and encapsulating the liquid-absorbing layer. Methods for manufacturing and using such composite wound dressings to effectuate healing of wounds on humans or other animals are described herein.

### CROSS-REFERENCE TO RELATED APPLICATIONS

This application claims priority to and benefit of U.S. Provisional Application Ser. No. 62/438,651, filed Dec. 23, 2016, entitled “Activated Carbon Composite Wound Dressing,” which is hereby incorporated herein by reference in its entirety.

### SUMMARY

Embodiments described herein include a composite wound dressing having a wound-interfacing layer including an activated carbon material. The composite wound dressing further includes a barrier material layer, a liquid-absorbing layer and a liquid-impermeable material outer layer. The composite wound dressing includes a bond substantially bonding a portion of the liquid-impermeable outer layer and the barrier material layer to the activated carbon material, where the bond forms a sealed pouch of un-bonded layers of the composite and encapsulates the liquid-absorbing layer.

In other embodiments, the instant disclosure is directed to methods for producing a composite wound dressing. The method includes providing a wound-interfacing layer including an activated carbon material and applying a barrier material layer to the wound-interfacing layer. A liquid-absorbing material layer is applied to the barrier material, and a liquid-impermeable outer layer is applied to the liquid-absorbing layer. At least a portion of the outer layer and the barrier material layer are bonded to the wound-interfacing layer to form a sealed pouch of un-bonded layers of the composite encapsulating the liquid-absorbing layer.

The instant disclosure further includes methods for treating a wound by contacting the wound with a composite wound dressing. The composite wound dressing includes a wound-interfacing layer including an activated carbon material. A barrier material layer is adhered to the wound-interfacing layer with a first adhesive. The composite wound dressing further includes a liquid-absorbing material layer and a liquid-impermeable material outer layer adhered to the liquid-absorbing material layer. A bond is included that substantially bonds a portion of the liquid-impermeable outer layer and the barrier material layer to the wound-interfacing layer. The bond forms a circumferentially sealed pouch of un-bonded layers of the composite and encapsulates the liquid-absorbing layer.

In another embodiment, the instant disclosure is directed to a composite wound dressing having a wound-interfacing layer including an activated carbon material. A barrier material layer is adhered to a wound-opposing side of the wound-interfacing layer with a first adhesive. A liquid-absorbing material layer is included and includes a liquid-absorbing pad. A liquid-impermeable material outer layer is adhered to the liquid-absorbing material layer with a second adhesive. A bond substantially bonds a portion of the liquid-impermeable outer layer and the barrier material layer to the wound-interfacing material layer to form a circumferentially sealed pouch of un-bonded layers of the composite and encompassing the liquid-absorbing layer.

### BRIEF DESCRIPTION OF THE DRAWINGS

For a fuller understanding of the nature and advantages of the present invention, reference should be made to the following detailed description taken in connection with the accompanying drawings, in which:

FIG. 1 is a schematic illustrating an embodiment of a composite wound dressing.

FIG. 2 is a graph illustrating the free swell capacity and retention capacity after loading of a number of cloths. The data labeled “Zorflex Plus” is an embodiment of a composite wound dressing in accordance with the present disclosure.

### DETAILED DESCRIPTION

Before the present compositions and methods are described, it is to be understood that they are not limited to the particular compositions, methodologies, or protocols described, as these may vary. It is also to be understood that the terminology used in the description is for the purpose of describing the particular versions or embodiments only, and is not intended to limit their scope, which will be limited only by the appended claims.

It must also be noted that as used herein and in the appended claims, the singular forms “a,” “an,” and “the” include plural references unless the context clearly dictates otherwise. Unless defined otherwise, all technical and scientific terms used herein have the same meanings as commonly understood by one of ordinary skill in the art. Although any methods and materials similar or equivalent to those described herein can be used in the practice or testing of embodiments disclosed, the preferred methods, devices, and materials are now described.

“Optional” or “optionally” means that the subsequently described event or circumstance may or may not occur, and that the description includes instances where the event occurs and instances where it does not.

As used herein, the term “about” means plus or minus 10% of the numerical value of the number with which it is being used. Therefore, about 50 mm means in the range of 45 mm to 55 mm.

Various embodiments of the invention are directed to a composite wound dressing including an activated carbon material, methods for preparing such composite wound dressings, and methods for treating wounds by applying the composite wound dressing of various embodiments to a wound.

According to an embodiment, the composite wound dressing includes an activated wound-interfacing layer including an carbon material. “Wound interfacing” as described herein shall mean that the activated carbon material of the wound-interfacing layer directly contacts, covers or otherwise faces the wound, and substantially no other layer of the composite wound dressing contacts, covers or otherwise directly faces the wound. In some embodiments, the activated carbon material includes woven cloth, non-woven cloth, knitted cloth, activated carbon felt, or combinations thereof. In one embodiment, the activated carbon material includes an activated carbon cloth. A suitable activated carbon cloth includes activated carbon cloth sold under the trade name Zorflex® (FM30K), and is commercially available from Calgon Carbon.

In some embodiments, the activated carbon material may include activated carbon particles, activated carbon powder, activated carbon fiber, or a combination of these materials. For example, in some embodiments, activated carbon particles may be immobilized or attached to a non-activated carbon based cloth and in other embodiments, activated carbon particles, powder, and/or fibers may be contained between sealed layers of a non-activated carbon based cloth. In still other embodiments, activated carbon particles, powder, and/or fibers may be included in a woven, non-woven, knitted, or felt activated carbon cloth material.

In some embodiments, the activated carbon material may include borax, i.e., sodium borate, sodium tetraborate, or disodium tetraborate. Without wishing to be bound by theory, the presence of borax in the activated carbon material may enhance the anti-microbial activity of the activated carbon in the absence of a known anti-microbial agent. Any amount of borax may be included in the activated carbon material. For example, in some embodiments, the borax may be about 0.001 wt. % to 50 wt. %, and in other embodiments, the borax may be about 0.01 wt. % to 30 wt. % or about 0.1 wt. % to 25 wt. %.

As discussed above, anti-microbial activity can be achieved in the absence of other known anti-microbial agents, because activated carbon inherently has antimicrobial activity, and this activity can be enhanced by the addition of borax. However, in certain embodiments, the activated carbon material may further include one or more anti-microbial agents other than activated carbon. For example, in some embodiments, the activated carbon material may include a noble metal such as silver, gold, palladium, platinum, copper, zinc, or a combination thereof, and in particular embodiments, the noble metal may be silver or zinc. In such embodiments, the noble metal may be provided at about 0.001 wt. % to about 30 wt. % or about 0.01 wt. % to about 10 wt. %.

In some embodiments, the noble metal may be provided as noble metal particles or powder, and in such embodiments, the particle size of the noble metal particles may be less than about 100 nm, less than 50 nm, and less than 25 nm. In embodiments, noble metal particles or powder are associated with the activated carbon in the activated carbon material, any means known in the art can be used to create such an association including, but not limited to, thermocracking, electroplating, electroless plating, or vacuum plating. In particular embodiments, noble metal particles or powder may be associated with activated carbon material, such as an activated carbon cloth, by immersion. For example, in some embodiments, activated carbon fibers or cloth may be immersed in a solution of silver nitrate for 1 to 720 minutes at a pH of 3 to 8, which reduces the silver allowing the silver to form particles on the surface of the activated carbon fiber or cloth. Such methods may further include drying the fiber or cloth to remove residual water, which is typically carried out at a temperature of from about 25° C. to about 150° C. Silver-carrying activated carbon fiber prepared according to the above process generally results in activated carbon fibers having a BET surface area of greater than about 400 m/g, carbon content of greater than about 50 wt %, silver content of greater than about 0.001 wt %, and a density of greater than about 1.8 g/m3.

In other embodiments, a therapeutically active agent can be included in the activated carbon material. The therapeutically active agent provided may be pre-adsorbed into the pore volume of the material and its subsequent release from the material may be controlled. In such embodiments, the release of the therapeutically active agent may be controlled by applying an electrical current to the material. Examples of suitable therapeutically active agents may include antibiotics, antimicrobials, sulfonamides, antiseptics, analgesics, or anesthetics and other medicaments and substances used to promote healing such as osmotic colloids, protease inhibitors, proteolytic enzymes, growth factors, steroidal or non-steroidal anti-inflammatory drugs, nutrients, antioxidants, and the like, and any combination of therapeutically active agents. Examples of particular active agents that may be useful in embodiments may include, but are not limited to, acrisorcin, haloprogin, iodochlorhydroxyquin, tolnaftate, triacetin, centella asiatica, econazole nitrate, mafenide, mupirocin, povidone iodine, chlohexidine, silver sulfadiazine, povidone iodine, silver salts, triclosan, sucralfate, quaternary ammonium salts, tetracycline, penicillins, terramycins, erythromycin, bacitracin, neomycin, polymycin B, mupirocin, clindamycin, and any mixtures thereof.

In some embodiments, the activated carbon material may further include one or more non-toxic, pharmaceutically, and dermatologically acceptable carriers, diluents, or excipients or combinations thereof. The therapeutically active agent and/or carriers, diluents, and/or excipients may be prepared for topical use and can be in various dosage forms including, but not limited to, a gel, a paste, an ointment, a cream, an emulsion, or a suspension. For example, a suitable thickener such as, aluminum stearate or hydrogenated lanolin, or gelling agent can be added to an aqueous or oil base to formulate an ointment or cream. Examples of suitable excipients include starch, tragacanth, cellulose derivative, polyethylene glycol, silicones, bentonite, silicic acid, talc, or a mixture thereof. In such embodiments, the activated carbon can be mixed with the therapeutically active agent, carrier, diluent, and/or excipient and other active components to provide the desired dosage form.

The composite wound dressing further includes a barrier material. The barrier material layer provides a barrier between the activated carbon material layer and a liquid-absorbing material layer, described below. In some embodiments, the barrier material layer permits the passage of exudate leaking from the wound to a liquid-absorbing layer, while substantially preventing the exudate from leaking back into the activated carbon material. In some embodiments, the barrier material layer may disperse the exudate and confine the exudate in a liquid-absorbing material layer. In one embodiment, the barrier material layer includes a polymer, such as a hydrophilic polymer. Examples of suitable polymers include, but are not limited to, polyethylene, polypropylene, polyester, polyamides such as nylons, fluoropolymers such as polyvinylidene fluoride (PVDF) or polytetrafluoroethylene (PTFE), ethylene methyl acrylate (EMA), and mixtures thereof. In other embodiments, the polymers may be drug-impermeable. Such drug impermeable materials include, for example, polyvinyl chloride, polyvinyl dichloride, polyurea, polyolefins, such, but not limited to, ethylene vinylacetate copolymer, polyethylene, and polypropylene, and polyesters, such as, but not limited to, polyethylene terephthlate, and the like and combinations and mixtures of these. In another embodiment, the barrier material layer includes a non-woven fabric.

In one embodiment, the barrier material layer is applied directly over the wound-interfacing layer on the wound-opposing side of the wound-interfacing layer. In another embodiment, the barrier material layer is applied to the wound-interfacing layer with an adhesive. An exemplary adhesive suitable for adhering the barrier material layer to the wound-interfacing layer includes a nonwoven web of a thermoplastic adhesive polymer resin. Other adhesives are further contemplated, and such adhesives would be apparent to one of skill in the art in view of this disclosure. Such other suitable adhesives may include any adhesive or combination of adhesives known in the medical arts. In general, the adhesive may be moisture vapor transmitting and pressure-sensitive, meaning that it forms a bond when applied with light pressure, of the type conventionally used for island-type wound dressings. Adhesives useful in various embodiments include, but are not limited to, acrylate ester copolymers, polyvinyl ethyl ether and polyurethane pressure sensitive adhesives. The relative thickness of the adhesive layer may vary among embodiments and may be optimized based on the type of adhesive used. In exemplary embodiments, the basis weight of the adhesive layer may be from about 20 g/m2 to about 250 g/m2 or from about 50 g/m2 to 150 g/m2. In particular embodiments, the adhesive may be a polyurethane-based pressure sensitive adhesive.

In some embodiments, the composite wound dressing includes a liquid-absorbing material layer. As contemplated herein, the liquid-absorbing material should be able to hold a significantly high volume of liquid, i.e., wound exudate, without leaking. For example, the liquid-absorbing material layer may have an absorbency of about 50 to about 2,000 grams of liquid per square foot of material. In another embodiment, the absorbency of the liquid-absorbing material layer may be from about 100 to about 1,500 grams per square foot. In one embodiment, the liquid-absorbing material layer comprises a sodium polyacrylate polymer dispersed between layers of cellulose paper. The liquid-absorbing material layer may have a weight of about 80 to about 300 grams per square meter. The configuration of the liquid-absorbing material layer in the composite wound dressing reduces gel leakage, and in some cases reduces gel leakage under pressure, when compared to commercially available wound dressings. In one embodiment, the liquid-absorbing material layer comprises a liquid-absorbing pad.

The composite wound dressing further includes a liquid-impermeable material outer layer. In one embodiment, the liquid-impermeable material is breathable and fluid repellant. In other words, the liquid-impermeable material may allow for the transfer of gases or air to the wound while preventing liquid or exudate from leaking through the liquid-impermeable material. A suitable material for the liquid-impermeable material includes polyethylene. In one embodiment, the liquid-impermeable material layer is a polyethylene spunbond fabric. In certain embodiments, the liquid-impermeable material is semi-permeable. For example, the liquid-impermeable material may be permeable to water vapor but not permeable to liquid. Suitable materials for liquid-impermeable material may have a moisture vapor transmission rate (MVTR) of, for example, about 300 g/m2/24 hrs. to about 5000 g/m2/24 hrs., or from about 500 g/m2/24 hrs. to about 2000 g/m2/24 hrs. at about 37° C. at 100% to 10% relative humidity. The liquid-impermeable material of such embodiments may further have a thickness of from about 10 μm to about 1000 μm or from about 100 μm to about 500 μm.

In one embodiment, an adhesive may be included to adhere the liquid-absorbing material layer to the liquid impermeable material outer layer. In one embodiment, this adhesive may include polyvinylpyrrolidone/vinyl acetate copolymer. Other adhesives may be employed and are discussed herein.

In one embodiment, a bond may be formed to substantially bond at least a portion of the barrier material layer and the liquid-impermeable material outer layer to the wound-interfacing layer. In another embodiment, the bond includes a circumferential seal between the barrier material layer and the liquid-impermeable outer layer with the wound-interfacing layer where the bond encompasses a substantially sealed pouch of un-bonded layers of the composite. In such an embodiment, the liquid-absorbing material layer is positioned in the pouch and is not bonded to the circumferential seal. “Circumferential” as used herein with reference to the bond or seal shall mean a bond or seal having a boundary in a continuous circuit. In one embodiment, the bond or seal is a thermal weld. The bond can be in any shape as desired, and the bond can take the desired shape of a finally formed composite wound dressing. Suitable shapes include, without limitations, square, rectangular, round, butterflied, or other desired shape.

The composite wound dressings of various embodiments can be used to treat any kind of wound such as, for example, lacerations, cuts, scrapes, abrasions, post-operative wounds, denuded skin, and burns, or other skin problems (e.g., allergies) and dressings of various sizes can be prepared such that minor wounds as well as larger wounds can be treated using the composite wound dressings of embodiments. In general, the composite dressing of such embodiments may allow transfer of air and moisture vapor into and out of the wound and trapping or capturing wound exudate while being liquid-impermeable and immobilizing microbes on the activated carbon. The composite wound dressings described herein can be used to treat wounds on humans or any other animal including, but not limited to, mammals, fish, reptiles, birds, and other creatures. Thus, medical and veterinary uses for the composite wound dressings described herein are encompassed by the invention, and such uses can be carried out by trained medical professionals, physicians, veterinarians, nurses, emergency medical technicians, and the like, or by consumers who purchase the wound dressings described herein over the counter.

In some embodiments, the composite wound dressing may be applied to a wound, such as those described above, directly. Therefore, embodiments include a composite wound dressing which can be applied to a wound, and the composite wound dressing may be shaped to adequately cover a wound. Such various shapes are described herein. In still further embodiments, the composite wound dressing designed and configured to be applied to a wound may include one or more additional components that are provided to, for example, improve healing, reduce adhesion to the wound, improve adherence to skin surrounding the wound, reduce itching, or otherwise aid in improving patient comfort. Additional components may include, without limitation, topical compositions including one or more therapeutic agents. Any type of topical composition including, but not limited to, gels, pastes, ointments, creams, emulsions, suspensions, or the like containing the therapeutic agent may be applied.

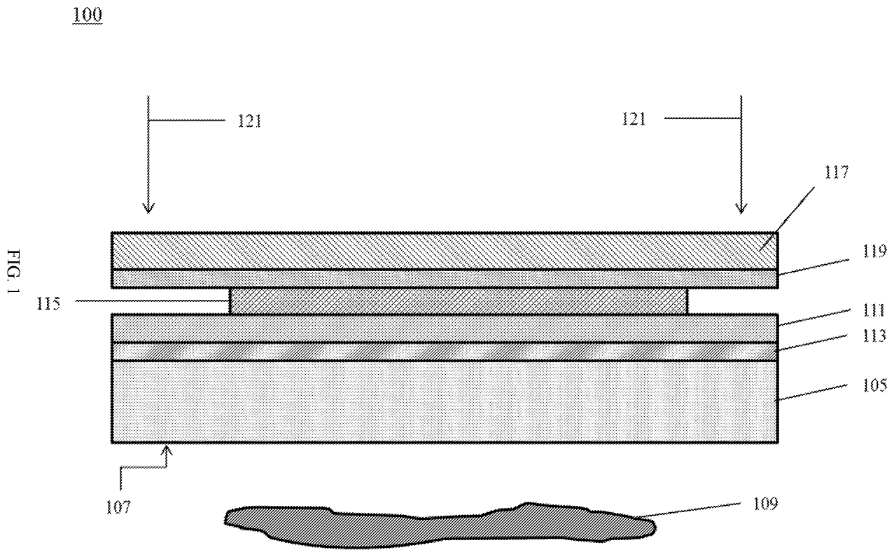

FIG. 1 illustrates an exemplary composite wound dressing 100. The composite wound dressing includes a first layer 105 having an activated carbon material. The activated carbon material includes activated carbon cloth sold under the trade name Zorflex® (FM30K). The first layer includes a wound interface 107 that directly interfaces, contacts, covers, or otherwise faces a wound 109. The composite 100 further includes a barrier layer 111 that is adhered to the first layer 105 by an adhesive layer 113. The adhesive layer 113 may be applied to either the wound opposing side of the first layer 105 or the wound facing side barrier layer 111. Once the adhesive layer 113 is applied, pressure, or other suitable action, may be used to aid in securing the barrier layer 111 to the first layer 105.

A liquid-absorbing pad 115 is provided on a wound-opposing side of the barrier layer 111. The barrier layer 111 allows the passage of liquid exudate from the wound 109 to the liquid-absorbing pad 115, but substantially prevents exudate captured by the liquid absorbing pad 115 from leaking back into the first layer 105 and/or back into the wound 109. The barrier layer 111 includes a hydrophilic non-woven material. The liquid-absorbing pad 115 includes a superabsorbent pad having a weight of about 80 to about 300 grams per square meter. In the exemplary embodiment of the composite wound dressing 100 of FIG. 1, the liquid absorbing pad 115 is a superabsorbent sodium polyacrylate polymer which is commercial available under the trade name Gelok®. The liquid-absorbing pad 115 may be positioned in the composite wound dressing 100 have a length shorter than the other layers within the composite wound dressing 100. This can allow the liquid-absorbing pad 115 to be entirely encapsulated in the composite wound dressing 100 after forming a seal or bond, as described below.

The outer layer 117 of the composite wound dressing 100 is a liquid-impermeable material that prevents liquid from entering the composite wound dressing 100 and prevents exudate capture by the liquid-absorbing pad 115 from leaking out of the composite wound dressing 100. The liquid-absorbing material includes a breathable material that can allow the passage of air and/or moisture vapor across the liquid-impermeable material while preventing the passage of liquids. The liquid-impermeable includes a polyethylene sheet or membrane. In the exemplary embodiment of the composite wound dressing 100 the outer layer 117 is adhered to the liquid-absorbing layer 115 by, optionally, an adhesive layer 119. The optional adhesive layer 119 can be applied to either the wound opposing side of the liquid-absorbing layer 115 or the wound-facing side of the outer layer 117. Once the adhesive layer 119 is applied, pressure, or other suitable action, may be used to aid in securing the liquid-absorbing layer 115 to the outer layer 117. The adhesive layer 119 includes a polyvinylpyrrolidone/vinyl acetate copolymer adhesive.

Once the layers are formed into a composite, the composite wound dressing 100 can be sealed to create a circumferential sealed bond or bead that may resemble the final shape of the composite wound dressing 100. A thermal heat weld may be used to create the sealed bead. In such an embodiment, a thermal weld head can be applied to contact and seal the composite wound dressing 100 along arrows 121. Although FIG. 1 illustrates a two-dimensional view of the wound dressing composite, it is contemplated that the thermal weld head can be in the a circumferential shape of the finally produced composite wound dressing. Suitable shapes are disclosed herein. The thermal weld head provides heat which melts and seals the outer layer 117, the barrier layer 111 and the adhesive layers 113, 119 to the first layer 105, thereby creating a bond to the activated carbon material of the first layer 105. The bond comprises a narrow sealing interface resulting in a sealed outer edge surrounding an unsealed pocket of the layers of the composite wound dressing 100. Thus, the layers of the composite wound dressing 100 within the areas sealed by the bond are unsealed and are encapsulated by the sealed bond. In the exemplary embodiment of FIG. 1, the liquid-absorbing layer 115 is not sealed upon contact with the thermal weld held, but, rather, remains encapsulated in the unsealed pocket. Once the composite wound dressing 100 is sealed by the thermal weld head, the excess unsealed material beyond the outer periphery of the bond can be trimmed to result in a finally formed composite wound dressing 100.

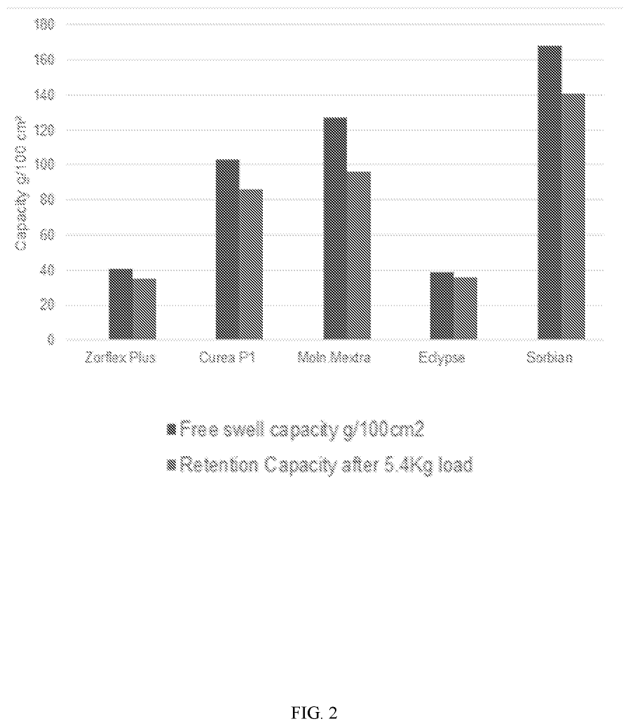

FIG. 2 illustrates the free swell capacity (in g/100 cm²) and retention capacity after a 5.4 Kg load for a number of cloths, including Zorflex Plus, Curea P1, Moln.Mextra, Eclypse, and Sorbian. The cloth labeled “Zorflex Plus” is an embodiment of a composite wound dressing in accordance with the present disclosure.

Generally, the composite wound dressings of various embodiments described above will be sterile and can be packaged in a microorganism-impermeable container such as a pouch. Further, the composite wound dressing of embodiments described herein provide against the leakage of liquid exudate from a wound and/or against gel leaking from the liquid-absorbing material under pressure conditions. The composite wound dressing are lightweight and flexible.

Certain embodiments herein further include methods of methods for producing a composite wound dressing. In one embodiment, the method includes providing a wound-interfacing layer including an activated carbon material. A barrier material is applied to the wound-opposing side of the wound-interfacing layer. The method includes applying an adhesive to secure the barrier material to the wound-interfacing layer. A liquid-absorbing material layer is applied to the wound opposing side of the barrier material. The liquid-absorbing material layer may include a super absorbent pad as described herein. A liquid-impermeable material outer layer is applied to the wound-opposing side of the liquid-absorbing material layer. An adhesive is applied to secure the liquid-absorbing material layer to the liquid-impermeable material layer. At least a portion of the outer layer and the barrier material layer and, optionally, the adhesive materials, are bonded to the wound-interfacing layer forming a sealed pouch of un-bonded layers of the composite. The bonding of the layers of the composite may be accomplished by a thermal weld. In some embodiments, the bonding includes forming a circumferential seal encompassing a sealed pouch of un-bonded layers of the composite. The sealed pouch may include the liquid-absorbing layer. The method may further include trimming excess layers outside periphery of the sealed pouch, resulting in a finally formed composite wound dressing. Embodiments herein describe suitable materials for each layer of the composite.

Embodiments further include methods for using the composite wound dressings described above. The methods of various embodiments generally include the step of contacting a wound with a composite wound dressing. The composite wound dressing including a wound-interfacing layer having an activated carbon material, a barrier material layer, which, optionally, may be adhered to the activated carbon material by an adhesive. The composite wound dressing further including an liquid-absorbing material layer positioned at a wound-opposing side of the barrier material, and a liquid-impermeable material outer layer positioned at a wound-opposing side of the liquid-absorbing material layer. The outer layer may, optionally, be adhered to the liquid-absorbing material layer with an adhesive. A circumferential bond is provided to seal between the liquid-impermeable outer layer and barrier material layer and, optionally, the adhesive layers to the wound-interfacing layer. In some embodiments, the bond encompasses a substantially sealed pouch of unbonded layers of the composite, where the pouch includes the liquid-absorbing layer.

The composite wound dressing and methods described herein can be used to treat any type of wound, including lacerations, cuts, scrapes, abrasion, post-operative wounds, denuded skin, burns, and the like. The composite wound dressing can remain in contact with the wound throughout the healing process. The composite wound dressings disclosed herein provide a dressing that can handle excessive amounts of exudation from a wound. Thus, wounds that are known to generate excessive amounts of exudate would benefit from the composite wound dressings due to the wound dressings ability to absorb the exudate, wick and keep the exudate away from the wound site. The anti-microbial activity of the activated carbon material in the wound-interfacing layer may also reduce the likelihood of infection by trapping and eradicating microbes in the wound at the time the composite wound dressing is applied and throughout the healing process by immobilizing microbes before they can reach the wound itself.

Although the present invention has been disclosed above, the disclosure does not limit the present invention. Persons having ordinary skill in the art can make any changes or modifications without departing from the spirit and scope of the present invention. Consequently, the scope of protection of the present invention is based on the claims attached.

1. A composite wound dressing comprising:
a wound-interfacing layer including an activated carbon material;
a barrier material later;
a liquid-absorbing material layer;
a liquid-impermeable outer layer; and
a bond substantially bonding a portion of the liquid-impermeable outer layer and the barrier material layer to the wound-interfacing layer, the bond forming a sealed pouch of un-bonded layers of the composite and encapsulating the liquid-absorbing layer.

2. The composite wound dressing of claim 1, wherein the activated carbon material comprises an activated carbon cloth.

3. The composite wound dressing of claim 1, wherein the activated carbon material comprises a woven cloth, non-woven cloth, knitted cloth, activated carbon felt, or combinations thereof.

4. The composite wound dressing of claim 1, wherein the activated carbon material comprises activated carbon particles, activated carbon powder, activated carbon fiber, or combinations thereof.

5. The composite wound dressing of claim 1, wherein the barrier material comprises a non-woven fabric.

6. The composite wound dressing of claim 1, wherein the liquid-absorbing material has an absorbency of about 75 to about 1,700 grams per square foot.

7. The composite wound dressing of claim 1, wherein the liquid-impermeable material comprises a polyethylene spunbond fabric.

8. The composite wound dressing of claim 1, further comprising a first adhesive disposed between the wound-interfacing layer and the barrier material layer.

9. The composite wound dressing of claim 8, wherein the first adhesive comprises a nonwoven web of a thermoplastic polymer resin.

10. The composite wound dressing of claim 1, further comprising a second adhesive disposed between the liquid-absorbing material layer and the liquid impermeable material outer layer.

11. The composite wound dressing of claim 10, wherein the second adhesive comprises a polyvinylpyrrolidone/vinyl acetate copolymer.

12. The composite wound dressing of claim 1, wherein the bond comprises a thermal weld.

13. The composite would dressing of claim 1, wherein the bond comprises a circumferential seal encompassing the sealed pouch.

14. A method for producing a composite wound dressing, the method comprising:
providing a wound-interfacing layer including an activated carbon material;
applying a barrier material to the wound-interfacing layer;
applying a liquid-absorbing material layer to the barrier material;
applying an outer layer including a liquid-impermeable material; and
bonding at least a portion of the outer layer and the barrier material layer to the wound-interfacing layer to form a sealed pouch of un-bonded layers of the composite and encapsulating the liquid-absorbing layer.

15. The method of claim 14, wherein the activated carbon material comprises activated carbon cloth.

16. The method of claim 14, wherein the activated carbon material comprises a woven cloth, non-woven cloth, knitted cloth, activated carbon felt, or combinations thereof.

17. The method of claim 14, wherein the activated carbon material comprises activated carbon particles, activated carbon powder, activated carbon fiber, or combinations thereof.

18. The method of claim 14, wherein the barrier material comprises a non-woven fabric.

19. The method of claim 14, wherein the liquid-absorbing material has an absorbency of about 75 to about 1,700 grams per square foot.

20. The method of claim 14, wherein the liquid impermeable material comprises a polyethylene spunbound fabric.

21. The method of claim 14, wherein applying the barrier material to the wound-interfacing layer further comprises applying a first adhesive.

22. The method of claim 21, wherein the first adhesive comprises a nonwoven web of thermoplastic polymer resin.

23. The method of claim 14, wherein applying the outer layer to the liquid-absorbing layer comprises applying a second adhesive.

24. The method of claim 23, wherein the second adhesive comprises a polyvinylpyrrolidone/vinyl acetate copolymer.

25. The method of claim 14, wherein the bonding comprises forming a thermal weld.

26. The method of claim 14, wherein the bonding comprises forming a circumferential seal encompassing the sealed pouch.

27. A method of treating a wound, the method comprising:
contacting a composite wound dressing with the wound, the composite wound dressing comprising:
a wound-interfacing layer including an activated carbon material;
a barrier material layer adhered to the activated carbon material with a first adhesive;
a liquid-absorbing material layer;
a liquid-impermeable material outer layer adhered to the liquid-absorbing layer with a second adhesive; and
a bond substantially bonding a portion of the liquid-impermeable outer layer and the barrier material layer to the wound-interfacing layer, the bond forming a circumferentially sealed pouch of un-bonded layers of the composite encapsulating the liquid-absorbing layer.

28. A composite wound dressing comprising:
a wound-interfacing layer including an activated carbon material;
a barrier material layer adhered to a wound-opposing side of the wound-interfacing layer with a first adhesive;
a liquid-absorbing material layer including a liquid-absorbing pad;
a liquid-impermeable material outer layer adhered to a wound-opposing side of the liquid absorbing material layer with a second adhesive; and
a bond substantially bonding a portion of the liquid-impermeable outer layer and the barrier material layer to the wound-interfacing material layer, the bond forming a circumferentially sealed pouch encapsulating the liquid absorbing material layer.
